(19) United States
(12) Patent Application Publication
(10) Pub. No.: US 2009/0076281 A1
(43) Pub. Date: Mar. 19, 2009

(54) PROCESS FOR THE PREPARATION OF LOSARTAN AND ITS SALTS

(75) Inventor: Carme Arnalot Aguilar, Girona (ES)

Correspondence Address: HOGAN & HARTSON LLP;IP GROUP, COLUMBIA SQUARE, 555 THIRTEENTH STREET, N.W., WASHINGTON, DC, 20004, US

(73) Assignee: MEDICHEM, S.A., Barcelona (ES)

(21) Appl. No.: 11/913,655

(22) PCT Filed: May 5, 2005
(86) PCT No.: PCT/IB2006/002878

§ 371 Date: Oct. 9, 2008

Related U.S. Application Data
(60) Provisional application No. 60/677,843, filed on May 5, 2005.

(51) Int. Cl. C07D 405/10

(52) U.S. Cl. 548/254

(57) ABSTRACT

The invention relates to the preparation of losartan and losartan potassium. More particularly, the invention relates to the preparation of losartan and losartan potassium in a simplified process that provides higher purity losartan potassium and losartan potassium having larger crystal sizes. The invention further includes formulating into readily usable dosage units for the therapeutic treatment (including prophylactic treatment) of mammals, including humans.

### CROSS REFERENCE TO RELATED APPLICATIONS

This application claims priority to U.S. Provisional Application No. 60/677,843, filed May 5, 2005, which application is expressly incorporated herein by reference in its entirety.

### BACKGROUND OF THE INVENTION

1. Field of the Invention

The invention relates, in general, to the preparation of losartan and its salts (e.g., losartan potassium). More particularly, the invention relates to the preparation of losartan and its salts (e.g, losartan potassium) in a simplified process that provides higher purity losartan potassium and losartan potassium having larger crystal sizes. The invention further includes formulating losartan, its salts (e.g., losartan potassium) and/or in vivo cleavable prodrugs thereof (collectively “the compounds of the invention”) into readily usable dosage units for the therapeutic treatment (including prophylactic treatment) of mammals, including humans.

2. Relevant Background

Losartan free acid is also known as 2-Butyl-4-chloro-1-[[(2′-(1H-tetrazol-5-yl)[1,1′-biphenyl]-4-yl]methyl]-1H-imidazole-5-methanol, whose potassium salt has been shown to be useful in the treatment of hypertension.

Losartan potassium has been approved by the FDA for the treatment of hypertension.

The product is marketed as coated tablets for oral administration under the name Cozaar®, and in combination with hydrochlorothiazide as coated tablets for oral administration under the trade mark of Hyzaar®.

Losartan may be prepared using the reactions and techniques described in U.S. Pat. No. 5,138,069, U.S. Pat. No. 5,130,439 and U.S. Pat No. 5,206,374, herein incorporated by reference.

Example 316 Part D of U.S. Pat. No. 5,138,069 discloses the isolation of losartan potassium from a mixture of isopropanol/heptane. According to the inventor's experience a very fine crystalline powder is obtained.

U.S. Pat. No. 5,859,258 discloses a method for crystallizing losartan potassium salt, which includes the addition of finely-milled losartan potassium.

One aspect of the present invention provides processes for preparation and crystallization of losartan potassium which directly produces large crystals with greatly improved stability, purity and good handling characteristics. These larger losartan potassium crystals can be formulated into tablets by direct compression.

According to the invention, the losartan potassium crystals obtained have a particle size distribution in which 90% of the particles have a diameter greater than about 45 μm and preferably greater than about 50 μm.

The processes of the invention are simple, cost-effective, and non-hazardous and are well suited for large-scale production. In this regard, when growing crystals a number of factors are important for producing a large crystal size. First, high purity of the ingoing losartan potassium is helpful in growing larger crystals. Also, conducting the crystallization just below reflux is helpful, and it is possible that dropping the temperature just below reflux decreases the amount of stress on the crystal. Additionally, using a slow rate of agitation further reduces crystal breakage.

### SUMMARY OF THE INVENTION

The invention provides a method for preparing losartan and its salts (e.g., losartan potassium). More particularly, the invention relates to the preparation of losartan and its salts (e.g., losartan potassium) in a simplified process that provides higher purity losartan potassium and losartan potassium having larger crystal sizes. This latter quality is particularly beneficial to formulating losartan potassium into usable dosage units. Thus, the invention further includes formulating losartan, its salts (e.g., losartan potassium) and/or in vivo cleavable prodrugs thereof (collectively “the compounds of the invention”) into readily usable dosage units for the therapeutic treatment (including prophylactic treatment) of mammals, including humans.

### DETAILED DESCRIPTION OF THE PREFERRED EMBODIMENTS

Reference will now be made in detail to the preferred embodiments of the invention. This invention may, however, be embodied in many different forms and should not be construed as limited to the embodiments set forth herein. In addition and as will be appreciated by one of skill in the art, the invention may be embodied as a method, system or process.

The invention provides an improved method for preparing losartan and its salts (e.g., losartan potassium). More particularly, the invention relates to the preparation of losartan and its salts (e.g., losartan potassium) in a simplified process that provides higher purity losartan potassium and losartan potassium having larger crystal sizes.

The ability to prepare losartan potassium having larger crystal sizes is particularly advantageous for formulating usable dosage units. Namely, higher purity, larger crystal losartan potassium can be readily formulated into tablets by direct compression. Thus, the invention further includes formulating losartan, its salts (e.g., losartan potassium) and/or in vivo cleavable prodrugs thereof (collectively “the compounds of the invention”) into readily usable dosage units for the therapeutic treatment (including prophylactic treatment) of mammals, including humans. Such formulations are normally formulated in accordance with standard pharmaceutical practice as a pharmaceutical composition. According to this aspect of the invention, there is provided a pharmaceutical composition that comprises the compounds of the invention, as defined hereinbefore, in association with a pharmaceutically acceptable diluent or carrier.

The compositions of the invention may be in a form suitable for oral use (e.g., as tablets, lozenges, hard or soft capsules, aqueous or oily suspensions, emulsions, dispersible powders or granules, syrups or elixirs), for topical use (e.g., as creams, ointments, gels, or aqueous or oily solutions or suspensions), for administration by inhalation (e.g., as a finely divided powder or a liquid aerosol), for administration by insufflation (e.g., as a finely divided powder) or for parenteral administration (e.g., as a sterile aqueous or oily solution for intravenous, subcutaneous, or intramuscular dosing or as a suppository for rectal dosing). For example, compositions intended for oral use may contain, for example, one or more coloring, sweetening, flavoring and/or preservative agents.

Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (e.g., polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (e.g., the sodium salt of benzoic acid, ethyl or propyl p-hydroxybenzoate), anti-oxidants (e.g., ascorbic acid), coloring agents, flavoring agents, and/or sweetening agents (e.g., sucrose, saccharine or aspartame).

Oily suspensions may be formulated by suspending the active ingredient in a vegetable oil (e.g., arachis oil, olive oil, sesame oil or coconut oil) or in a mineral oil (e.g., liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (e.g., sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedures well known in the art.

Compositions for administration by insufflation may be in the form of a finely divided powder containing particles of average diameter of, for example, 30 μm or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used, and the aerosol device is conveniently arranged to dispense a metered quantity of active ingredient.

The amount of a compound of this invention that is combined with one or more excipients to produce a single dosage form will necessarily vary depending upon the host treated and the particular route of administration. For example, a formulation intended for oral administration to humans will may contain, for example, from 0.5 mg to 2 g of active ingredient compounded with an appropriate and convenient amount of excipients which may vary from about 5 to about 98 percent by weight of the total composition. Dosage unit forms will generally contain about 1 mg to about 500 mg of an active ingredient.

The size of the dose for therapeutic or prophylactic purposes of the compounds of the invention will naturally vary according to the nature and severity of the conditions, the age and sex of the animal or patient, and the route of administration, according to well known principles of medicine. For example, the method may comprise at least one of an hourly administration, a daily administration, a weekly administration, or a monthly administration of one or more compositions described herein.

In addition to the compounds of the invention, the invention also includes solvates, pharmaceutically acceptable prodrugs, pharmaceutically active metabolites, and pharmaceutically acceptable salts of such compounds.

The term “solvate” refers to an aggregate of a molecule with one or more solvent molecules.

A “pharmaceutically acceptable prodrug” is a compound that may be converted under physiological conditions or by solvolysis to the specified compound or to a pharmaceutically acceptable salt of such compound.

A “pharmaceutically active metabolite” is a pharmacologically active product produced through metabolism in the body of a specified compound or salt thereof. Metabolites of a compound may be identified using routine techniques known in the art, and their activities determined using tests such as those described herein.

Prodrugs and active metabolites of a compound may be identified using routine techniques known in the art. Various forms of prodrugs are known in the art.

According to the invention, suitable methods of administering the therapeutic composition of the invention to a patient include any route of in vivo administration that is suitable for delivering the composition into a patient. The preferred routes of administration will be apparent to those of skill in the art, depending on the type of condition to be prevented or treated, and/or the target cell population. Preferred methods of in vivo administration include, but are not limited to, intravenous administration, intraperitoneal administration, intramuscular administration, intranodal administration, intracoronary administration, intraarterial administration (e.g., into a carotid artery), subcutaneous administration, transdermal delivery, intratracheal administration, intraarticular administration, intraventricular administration, inhalation (e.g., aerosol), intracranial, intraspinal, intraocular, intranasal, oral, bronchial, rectal, topical, vaginal, urethral, pulmonary administration, impregnation of a catheter, and direct injection into a tissue.

It will be apparent to those skilled in the art that various modifications and variations can be made in the invention and specific examples provided herein without departing from the spirit or scope of the invention. Thus, it is intended that the invention covers the modifications and variations of this invention that come within the scope of any claims and their equivalents.

### SPECIFIC EXAMPLES

The following examples are for illustrative purposes only and are not intended, nor should they be interpreted, to limit the scope of the invention.

General Experimental Conditions:

i. HPLC Method:

The chromatographic separation was carried out in a Kromasil C18 4.6×250 mm, 5 μm I.D column.

The mobile phase was prepared by mixing 350 ml of acetonitrile with 650 ml of pH=3.0 buffer, which was prepared from 8.62 g of ammonium dihydrogen orthophosphate in 1000 mL of water adjusting the pH to 3.0 with 10% orthophosphoric acid. This mobile phase was mixed and filtered through 0.22 μm nylon filter under vacuum.

The chromatograph was equipped with a 254 nm detector, and the flow rate was 1.2 ml per minute. Test samples (20 μL) were prepared by dissolving the appropriate amount of sample in order to obtain 1.0 mg/mL of a 7:3 mixture of acetonitrile and water.

ii. Particle Size Measurement

The particle size for losartan potassium was measured using a Malvern Mastersizer S particle size analyzer with an MS1 Small Volume Recirculating unit attached. A 300RF mm lens and a beam length of 2.4 mm were used. Samples for analysis were prepared by dispersing a weighed amount of losartan potassium (approx. 0.05 g) in 20 mL of 5% of Span 85 in Hexane. The suspension was sonicated for 3 minutes and delivered drop-wise to a background corrected measuring cell previously filled with 5% of Span 85 in Hexane until the obscuration reached the desired level. Volume distributions were obtained for three times. Upon measurement completion, the sample cell was emptied and cleaned, refilled with suspending medium and the sampling procedure repeated again. For characterization, the values of D₁₀, D₅₀ and D₉₀ (by volume) were specifically listed, each one being the mean of the six values available for each characterization parameter.

### Example 1

### Preparation of Losartan Potassium Salt

Step 1: Preparation of Losartan Free Acid (2-Butyl-4-chloro-1-[[(2′-(1H-tetrazol-5-yl)[1,1′-biphenyl]-4-yl]methyl]-1H-imidazole-5-methanol)

According to one aspect of the invention, losartan (free acid) can be prepared by treating 2-butyl-4-chloro-1-[[2′-(2-trityl-2H-tetrazol-5-yl)[1,1′-biphenyl]-4-yl]methyl]-1H-imidazole-5-methanol with hydrochloric acid in a ketonic solvent (preferably acetone) at an elevated specific temperature. A preferred temperature range is between room temperature and reflux temperature and preferably not more than 45° C. The reaction is carried out at this elevated for a period of time between 3 to 7 hours and preferably for about 5 hours.

After treatment at the elevated temperature, potassium hydroxide is added to the reaction mixture, and the ketonic solvent is removed by distillation. Next, an acid (preferably hydrochloric acid) is added to adjust the pH to within a range of approximately 1.8 to 2.2. The solution is then filtered to separate the solid, which can then optionally be suspended in water and/or dried under reduced pressure at an elevated temperature (e.g., 40-50° C.).

Step 2: Conversion of Losartan (Free Acid) into Losartan Potassium Salt (Crude)

The losartan free acid produced in the previous step is next dissolved in a ketonic solvent (preferably methyl ethyl ketone), and a solution of potassium hydroxide dissolved in isopropyl alcohol is added to adjust the pH of the solution to within a range of approximately 8.0 to 9.2. Optionally, the solution can be filtered to remove any insoluble materials and/or treated with a decolorizing agent.

Next, the pH of the solution is adjusted to within a range of approximately 10.8 to 11.2 by the addition of a solution of potassium hydroxide dissolved in isopropyl alcohol. The solution is then distilled in order to obtain a reaction mixture with an approximate water content of 0.06%. Crude losartan potassium salt is then isolated from the solution, which can optionally be dried under reduced pressure at an elevated temperature (e.g., approximately 30-50° C., preferably at about 40° C.).

Step 3: Process for Obtaining Large Crystal Losartan Potassium Salt

The losartan potassium salt prepared in the previous step is next dissolved in an alcohol solvent (e.g., isopropyl alcohol and/or methanol) and heated, as in the first step, to reflux temperature. Heptane is added to this solution, and the solution is allowed to cool to room temperature.

From the room temperature solution, large size crystals of losartan potassium are isolated. The isolated crystals can optionally be recrystallized. Thereafter, the losartan potassium crystals are dried under reduced pressure at an elevated temperature (e.g., approximately 30-50° C., preferably at about 40° C.).

### Example 2

### Preparation of Losartan Potassium Salt

Step 1: Preparation of Trityl Losartan (2-butyl-4-chloro-1-{[2′-(2-trityl-2H-tetrazol-5yl)biphenyl-4-yl]-methyl}-1H-imidazol-5-yl)methanol

In a suitable reactor, 92.79 g of 5-[4′-(bromomethyl)-1,1′-bimethyl-2-yl]-2-(triphenylmethyl)-2H-tetrazole, 195 mL of dimethylformamide, 31.06 g of 2-butyl-4-chloro-1H-imidazole-5-carbaldehyde and 27.37 g of potassium carbonate were combined. The resulting suspension was stirred at 20-22° C. for 3 hours. Thereafter, 12.68 mL of an aqueous solution of sodium borohydride (20%) was added to the solution over approximately 5 minutes. The suspension was then stirred and heated to 48-52° C. for 3 hours. After the reduction, the mixture was cooled to 20-25° C. The mixture was then combined over approximately 20 minutes with 117 mL of ethyl acetate and a previously prepared solution of 390 mL of water and 10.3 mL of acetone. The resulting suspension obtained was stirred for 2 hours. Thereafter the suspension was filtered, and the resulting solid was washed with 40 mL of water.

The resulting wet solid was next placed in a suitable reactor with 300 mL of ethyl acetate. The suspension was then heated to reflux temperature until a solution was obtained, and the mixture was cooled to 0-5° C. for 2 hours. The suspension was then filtered, and the solid was washed with 15 mL of ethyl acetate to yield 148.05 g of wet (2-butyl-4-chloro-1-{[2′-(2-trityl-2H-tetrazol-5yl)biphenyl-4-yl]-methyl}-1H-imidazol-5-yl)methanol (trityl losartan) (estimated dry mass: 109.9 g, yield 99.2%).

Step 2: Preparation of Losartan Free Acid (2-butyl-4-chloro-1-[[2′-(1H-tetrazol-5-yl)[1,1′-biphenyl]-4-yl]methyl]-1H-imidazole-5-methanol)

In a suitable reactor, 125.5 g of wet (2-butyl-4-chloro-1-{[2′-(2-trityl-2H-tetrazol-5yl)biphenyl-4-yl]-methyl}-1H-imidazol-5-yl)methanol (estimated dry mass: 99.8), 385 mL of acetone and 23.1 mL of hydrochloric acid were combined at room temperature. The resulting solution was stirred and heated to 40° C. and maintained at this temperature for approximately 5 hours.

The resulting suspension was cooled at room temperature, and 14.75 g of potassium hydroxide was added. Next, approximately 50% of the volume of acetone was removed by distillation under vacuum, while preferably not raising the internal temperature above 30° C. The suspension was then cooled to 10-15° C. and was charged with 330 mL of water over about 30 minutes. Next, the pH was adjusted using hydrochloric acid to within the range of 1.8-2.2. The suspension was then filtered, and the wet solid was stirred in a suitable reactor with 200 mL of water. The obtained suspension was stirred for 30 minutes, filtered, and the resulting solid was washed with 2×40 mL of water to yield 95.53 g of losartan free acid (estimated dry mass 86.06 g).

Step 3 Conversion of Losartan (Free Acid) into Losartan Potassium Salt

In a suitable reactor, 87.26 g of wet losartan free acid (estimated dry mass 78.79 g) produced in the previous step was dissolved in 850 mL of methyl ethyl ketone (MEK). The pH was then adjusted to within 10.8-11.2 by addition of a solution of potassium hydroxide dissolved in isopropyl alcohol. The solution was then distilled in order to obtain a reaction mixture with an approximate water content of 0.60%. The mixture was then cooled to 0-5° C. and filtered to yield 44.04 g of crude losartan potassium potassium salt.

In a suitable reactor, 43.03 g of the crude losartan potassium salt was combined with 155 mL of isopropyl alcohol and 3 mL of methanol. The resulting white suspension was stirred and heated to reflux temperature (˜82° C.). After cooling, 77 mL of heptane was added, and the solution was cooled to 0-5° C. for at least 2 hours. The suspension was then filtered, and the obtained solid was washed with 15 mL of isopropyl alcohol. The wet solid obtained was then placed in a suitable reactor with 160 mL of methanol, and the solution was then filtered and distilled. Next, 30 mL of isopropyl alcohol was added, and the solution was distilled again.

The wet solid was next placed in a suitable reactor with 155 mL of isopropyl alcohol and 3 mL of methanol. The white suspension was stirred and heated to reflux temperature.

After cooling, it was combined with 77 mL of heptane and cooled to 0-5° C. for at least 2 hours. The suspension was then filtered, and the solid obtained was washed with 15 mL of isopropyl alcohol. Thereafter, the losartan potassium crystals were dried at 60° C., to constant mass, to yield 35.23 g (55.5%). Analytical data: HPLC purity: 99.93%, Assay with HClO4: 99.8%.

### Example 3

### Preparation of Losartan Potassium from Trityl Losartan (2-butyl-4-chloro-1-{[2′-(2-trityl-2H-tetrazol-5yl)biphenyl-4-yl]-methyl}-1H-imidazol-5-yl)methanol)

In an inertized 630 L glass-lined reactor, 52 kg (78.2 mol) of trityl losartan, 144 kg of acetone and 13.09 kg (125.6 mol) of 35% hydrochloric acid were combined. The mixture was stirred at about 40° C. for 5 hours. Thereafter, the mixture was cooled to 20-25° C., and 8.24 kg (124.8 mol) of 85% potassium hydroxide and 18.9 kg of water were added. The mixture was then distilled under vacuum without exceeding 30° C. Next, the temperature was lowered to 10-15° C., and 225 kg of water was added. While maintaining the temperature at 10-15° C., the pH of the mixture was adjusted to 1.8-2.2, and the mixture was filtered. The collected solid was then washed twice with 20 kg of water.

The solid was then combined in a reactor with 135 kg of water. The mixture was then stirred for 30 minutes at 20-25° C., and the pH was adjusted to 1.8-2.2, as necessary. The mixture was then filtered, and the collected solid was washed twice with 20 kg of water. The solid was then combined in a reactor with 391 kg of methyl ethyl ketone, to which was then added a mixture of potassium hydroxide in isopropyl alcohol until the pH was 10.8-11.2.

Next, the mixture was distilled under atmospheric pressure until 230 kg was removed. Once the distillation was complete, a further 230 kg of methyl ethyl ketone were added. The mixture was then stirred for 2 hours at 10-15° C. and filtered. The collected solid was then washed twice with 12 kg of methyl ethyl ketone.

The solid was then combined in a suitable reactor with 69.2 kg of isopropyl alcohol and 1.4 kg of methanol. The resulting suspension was heated to reflux at atmospheric pressure, and then 29.6 kg of heptane was added over 30 minutes. The mixture was then cooled to 0-5° C. over a minimum of 2 hours, and stirred at this temperature for a minimum of 2 hours. The suspension was then filtered, and the collected solid was washed with 7 kg of isopropyl alcohol.

The solid was then combined in a suitable reactor with 72.3 kg of methanol, and the mixture was stirred at 20-25° C. until complete dissolution occurred. The resulting solution was then filtered and distilled at atmospheric pressure until 62 kg of methanol were collected. To the remaining residue, 12.5 kg of isopropyl alcohol was added, and the mixture was distilled under vacuum to dryness. This process was then repeated a second time.

Next, 69.2 kg of isopropyl alcohol and 1.4 kg of methanol were added to the residue, and the mixture was heated to reflux at atmospheric pressure. At reflux temperature, 29.6 kg of heptane were added to the mixture over 15-30 minutes. The mixture was then cooled to 0-5° C. over a minimum of 2 hours and stirred at this temperature for a minimum of 2 hours. The suspension was then filtered, and the collected solid was washed with 7 kg of isopropyl alcohol. The collected solid was dried at 60° C. under vacuum for 5 hours to yield 20.26 kg of losartan potassium (Yield: 56.3%). Analytical data: HPLC purity: 99.96%; Assay with HClO₄: 99.62%; particle size (by volume) (Malvern): D₁₀20.1 μm, D₅₀ 82.6 μm, D₉₀,169.2μm; particle size after sieving (by volume) (Malvern): D₁₀ 17.3 μm, D₅₀ 81.8 μm, D₉₀ 156.8 μm.

### Example 4

### Preparation of Losartan Potassium Salt Dosage Units

Losartan potassium prepared according to the preceding examples can be formulated (e.g., packed) so as to prevent hydration and/or in a manner to improve stability during the storage or transport. Such formulation improvements can be achieved by storing the active substance in an inert atmosphere, which is technologically and economically simple. For example, the active substance is stored in polythene bags or containers. The inert atmosphere can be achieved using nitrogen, argon or a mixture thereof. Similarly, silica gel and/or other desiccant materials in air permeable bags may optionally be used in conjunction with an inert gas. Such inert gases include those that pose no health risks, which do not react with losartan potassium and are free from moisture.

Although the invention has been described and illustrated with a certain degree of particularity, it is understood that the present disclosure has been made only by way of example, and that numerous changes in the conditions and order of steps can be resorted to by those skilled in the art without departing from the spirit and scope of the invention.

What is claimed is:

1. A process for converting losartan to losartan potassium comprising:
preparing a mixture of losartan free acid and an organic solvent;
adding a solution of potassium hydroxide in an alcohol to the mixture until a pH of approximately 11 is achieved;
distilling water from the mixture until a residual water content of approximately 0.6% (w/w) is obtained; and
filtering the mixture to obtain losartan potassium.

2. The process of claim 1, wherein the organic solvent is methyl ethyl ketone.

3. The process of claim 1, wherein the alcohol is isopropyl alcohol.

4. A process for preparing losartan potassium having a large crystal size comprising:
preparing a first solution of 2-butyl-4-chloro-1-[[2′-(2-trityl-2H-tetrazol-5-yl)[1,1′-biphenyl]-4-yl]methyl]-1H-imidazole-5-methanol and hydrochloric acid in a ketonic solvent at an elevated temperature to form a solution of losartan free acid;
treating the losartan free acid solution with a solution of potassium hydroxide dissolved in a first alcohol solvent;
removing the ketonic solvent from the losartan free acid solution;
adjusting the pH of the losartan free acid solution;
isolating losartan free acid;
preparing a second solution by dissolving losartan free acid in a second ketonic solvent;
adding a solution of potassium hydroxide in isopropyl alcohol to the second solution to adjust the pH to approximately 8.0 to approximately 11.2;
distilling the second solution until the solution has a water content of approximately 0.06%;
isolating crude losartan potassium;
preparing a third solution of crude losartan potassium in a second alcohol solvent;
heating the third solution to reflux temperature;
adding an alkane solvent to the third solution to form a ternary solvent system;
allowing the ternary solvent system to cool; and
isolating losartan potassium.

5. The process of claim 4 further comprising drying the obtained losartan potassium.

6. The process of claim 4, wherein the first and second alcohol solvents are each at least one of isopropyl alcohol, methanol and combinations thereof.

7. The process of claim 4, wherein the first alcohol solvent is isopropyl alcohol.

8. The process of claim 4, wherein the second alcohol solvent is methanol.

9. The process of claim 4, wherein the alkane solvent is heptane.

10. The process of claim 4, wherein the first alcohol solvent is isopropyl alcohol, the second alcohol solvent is methanol and the alkane solvent is heptane.

11. The process of claim 10, wherein the ternary solvent system comprises approximately 66-74 % by weight of isopropyl alcohol, approximately 0.8-2% by weight of methanol and approximately 24-35% by weight of heptane.

12. The process of claim 4, where said step of allowing the ternary solvent system to cool comprises cooling the temperature from approximately reflux temperature to approximately 0-5° C. over approximately 2 hours.

13. A process for preparing losartan from (2-butyl-4-chloro-1-{[2′-(2-trityl-2H-tetrazol-5yl)biphenyl4-yl]-methyl}-1H-imidazol-5-yl)methanol) (trityl losartan) comprising:
combining trityl losartan in an acidic solvent system comprising an acid solution and at least one organic solvent to form a reaction mixture;
reacting the trityl losartan in the reaction mixture;
distilling the reaction mixture to remove the at least one organic solvent;
adding water to the reaction mixture;
adjusting the pH of the reaction mixture to between approximately 1.8 and 2.2; and
isolating solid losartan from the reaction mixture.

14. The process of claim 13 further comprising suspending the isolated solid losartan in water and filtering.

15. The process of claim 13 further comprising converting losartan to losartan potassium.

16. A process for preparing losartan potassium salt having large crystal sizes comprising:
reacting crude losartan potassium in a ternary solvent system comprising at least two different alcohol solvents and an alkane solvent.

17. The process of claim 16, wherein the at least two different alcohol solvents are at least two of isopropyl alcohol, methanol and combinations thereof

18. The process of claim 16, wherein the alkane solvent is heptane.

19. The process of claim 16, wherein said ternary solvent system comprises approximately 66-74% by weight of isopropyl alcohol, approximately 0.8-2% by weight of methanol and approximately 24-35% by weight of heptane.

20. The process of claim 4 further comprising filtering at least one of the third solution and the ternary solvent system to remove insoluble particles.

21. The process of claim 4 further comprising:
distilling at least one of the third solution and the ternary solvent system to remove the second alcohol solvent;
adding isopropyl alcohol; and
recrystallizing losartan potassium in a mixture comprising at least one of isopropyl alcohol, methanol and heptane.

22. The process of claim 16 further comprising filtering at least one of the third solution and the ternary solvent system to remove insoluble particles.

23. The process of claim 16 further comprising:
distilling at least one of the third solution and the ternary solvent system to remove the second alcohol solvent;
adding isopropyl alcohol; and
recrystallizing losartan potassium in a mixture comprising at least one of isopropyl alcohol, methanol and heptane.

24-29. (canceled)
